(19) United States
(12) Patent Application Publication
(10) Pub. No.: US 2026/0269075 A1
(43) Pub. Date: Sep. 10, 2026

(54) HEALTH CONDITION ESTIMATION SYSTEM, INFORMATION PROCESSING APPARATUS, HEALTH CONDITION ESTIMATION METHOD, AND PROGRAM

(71) Applicant: Yokogawa Electric Corporation, Tokyo (JP)

(72) Inventors: Kiyomi Muraoka, Musashino-shi (JP); Hideo Uemura, Musashino-shi (JP); Kie Shimamura, Musashino-shi (JP)

(73) Assignee: Yokogawa Electric Corporation, Tokyo (JP)

(21) Appl. No.: 19/556,073

(22) Filed: Mar. 4, 2026

(30) Foreign Application Priority Data
Mar. 6, 2025 (JP) 2025-035920

(51) Int. Cl. G16H 50/30; G06F 3/01; G16H 20/70; G16H 40/67; G16H 50/20

(52) U.S. Cl. CPC G16H 50/30; G06F 3/015; G16H 20/70; G16H 40/67; G16H 50/20

(57) ABSTRACT

A health condition estimation system (1) according to the present disclosure includes a brain wave sensor (10) configured to detect brain waves of a user and output brain wave information and an information processing apparatus (20). The information processing apparatus (20) includes a processor (25) configured to estimate a health condition of the user, based on the brain wave information and operation information from when the user operates the information processing apparatus (20), and output information on the health condition of the user. This improves the accuracy of estimation of a health condition based on biological information.

### CROSS-REFERENCE TO RELATED APPLICATION

The present application claims priority to Japanese Patent Application No. 2025-035920 filed on Mar. 6, 2025, the entire contents of which are incorporated herein by reference.

### TECHNICAL FIELD

The present disclosure relates to a health condition estimation system, an information processing apparatus, a health condition estimation method, and a program.

### BACKGROUND

Techniques exist for estimating a health condition based on biological information measured by a sensor.

For example, Patent Literature (PTL) 1 discloses a technique for determining whether a person to be measured is in a resting state and calculating a condition value related to the physical condition of the person to be measured based on sensor information measured when the person to be measured is in a resting state.

### Citation List

### Patent Literature

PTL 1: WO 2018/016459 A1

### SUMMARY

A health condition estimation system comprising:

a brain wave sensor configured to detect brain waves of a user and output brain wave information; and

an information processing apparatus, wherein

the information processing apparatus comprises a processor configured to estimate a health condition of the user, based on the brain wave information and operation information from when the user operates the information processing apparatus, and output information on the health condition of the user.

An information processing apparatus for estimating a health condition of a user, the information processing apparatus comprising a processor configured to:

acquire brain wave information from a brain wave sensor that detects brain waves of a user and outputs the brain wave information; and

estimate a health condition of the user, based on the brain wave information and operation information from when the user operates the information processing apparatus, and output information on the health condition of the user.

A health condition estimation method comprising:

acquiring brain wave information from a brain wave sensor configured to detect brain waves of a user; and

estimating a health condition of the user, based on the brain wave information and operation information from when the user operates an information processing apparatus, and outputting information on the health condition of the user.

A program configured to cause a computer to perform operations comprising:

acquiring brain wave information from a brain wave sensor configured to detect brain waves of a user; and

estimating a health condition of the user, based on the brain wave information and operation information from when the user operates an information processing apparatus, and outputting information on the health condition of the user.

### BRIEF DESCRIPTION OF THE DRAWINGS

In the accompanying drawings:

FIG. 1 is a diagram illustrating a schematic configuration of a health condition estimation system according to an embodiment;

FIG. 2 is a diagram illustrating how a health condition is estimated using an estimation model; and

FIG. 3 is a flowchart illustrating an example of the operation of the health condition estimation system according to an embodiment.

### DETAILED DESCRIPTION

Demand exists for improving the estimation accuracy when estimating a health condition based on biological information.

It would be helpful to provide a health condition estimation system, an information processing apparatus, a health condition estimation method, and a program that are capable of improving estimation accuracy in estimating a health condition based on biological information.

Some embodiments of the present disclosure are illustrated below.

[1] A health condition estimation system comprising:

a brain wave sensor configured to detect brain waves of a user and output brain wave information; and

an information processing apparatus, wherein

the information processing apparatus comprises a processor configured to estimate a health condition of the user, based on the brain wave information and operation information from when the user operates the information processing apparatus, and output information on the health condition of the user.

According to such a health condition estimation system, the accuracy of estimation of a health condition based on biological information can be improved.

[2] In the health condition estimation system according to [1],

the information processing apparatus may further comprise a memory configured to store an estimation model created by machine learning based on training data,

the estimation model may be a model configured to output the information on the health condition when the brain wave information and the operation information are inputted, and

the processor may be configured to output the information on the health condition by inputting the brain wave information and the operation information into the estimation model.

With this configuration, the health condition of the user can be estimated using an estimation model constructed by machine learning.

[3] In the health condition estimation system according to [1] or [2],

the information processing apparatus may further comprise an input interface configured to accept an operation by the user, and

the processor may be configured to acquire, as the operation information, an operation by the user on the input interface.

With this configuration, operations by the user on the input interface can be acquired as operation information.

[4] In the health condition estimation system according to any one of [1] to [3],

the input interface may include at least one of a keyboard, a mouse, and a touchscreen.

With this configuration, operations by the user on the keyboard, mouse, or touchscreen can be acquired as operation information.

[5] In the health condition estimation system according to any one of [1] to [4],

the information on the health condition may include a stress level of the user.

With this configuration, the stress level of the user can be estimated.

[6] In the health condition estimation system according to any one of [1] to [5],

the information processing apparatus may further comprise an output interface, and

the processor may be configured to generate an action proposal to propose to the user based on the information on the health condition and display the generated action proposal on the output interface.

With this configuration, appropriate actions can be proposed to the user according to the health condition of the user.

[7] In the health condition estimation system according to any one of [1] to [6],

the processor may be configured to acquire a schedule of the user and generate the action proposal based on the acquired schedule and the information on the health condition.

With this configuration, appropriate actions can be proposed to the user according to the health condition of the user, taking into account the schedule of the user.

[8] An information processing apparatus for estimating a health condition of a user, the information processing apparatus comprising a processor configured to:

acquire brain wave information from a brain wave sensor that detects brain waves of a user and outputs the brain wave information; and

estimate a health condition of the user, based on the brain wave information and operation information from when the user operates the information processing apparatus, and output information on the health condition of the user.

According to such an information processing apparatus, the accuracy of estimation of a health condition based on biological information can be improved.

[9] A health condition estimation method comprising:

acquiring brain wave information from a brain wave sensor configured to detect brain waves of a user; and

estimating a health condition of the user, based on the brain wave information and operation information from when the user operates an information processing apparatus, and outputting information on the health condition of the user.

According to such a health condition estimation method, the accuracy of estimation of a health condition based on biological information can be improved.

[10] A program configured to cause a computer to perform operations comprising:

acquiring brain wave information from a brain wave sensor configured to detect brain waves of a user; and

estimating a health condition of the user, based on the brain wave information and operation information from when the user operates an information processing apparatus, and outputting information on the health condition of the user.

According to such a program, the accuracy of estimation of a health condition based on biological information can be improved.

According to the present disclosure, a health condition estimation system, an information processing apparatus, a health condition estimation method, and a program that are capable of improving estimation accuracy in estimating a health condition based on biological information can be provided.

An embodiment of the present disclosure will be described below, with reference to the drawings.

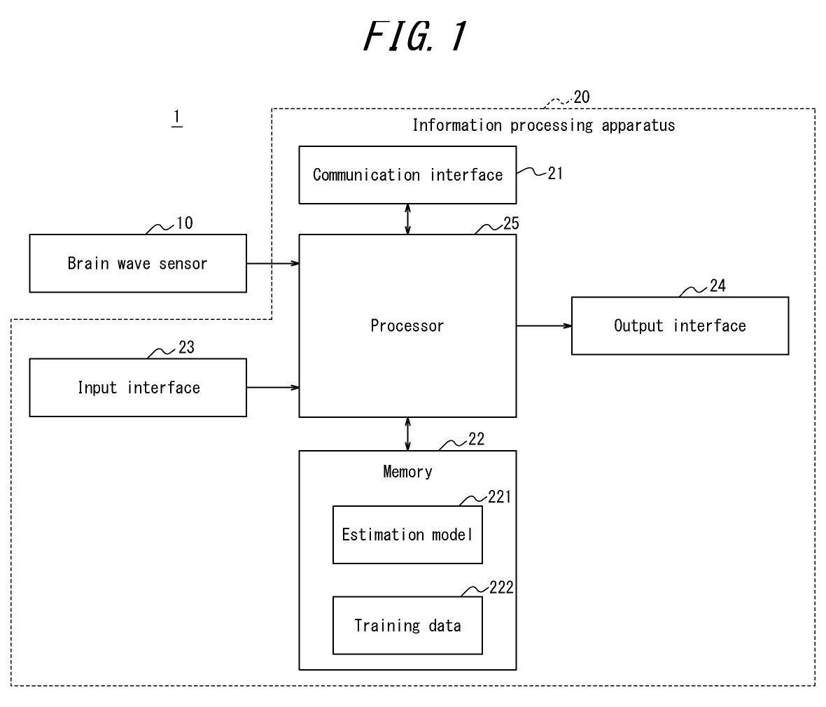

FIG. 1 is a diagram illustrating a schematic configuration of a health condition estimation system 1 according to an embodiment of the present disclosure. The health condition estimation system 1 includes a brain wave sensor 10 and an information processing apparatus 20. The brain wave sensor 10 and the information processing apparatus 20 can communicate by wireless communication or wired communication.

The health condition estimation system 1 can estimate the health condition of a user who operates the information processing apparatus 20. The user may be a desk worker who operates the information processing apparatus 20 for work, but the user is not limited to this example.

The brain wave sensor 10 is a sensor that detects brain waves of a user operating the information processing apparatus 20 and outputs brain wave information. Upon detecting brain waves of the user, the brain wave sensor 10 transmits brain wave information to the information processing apparatus 20.

The brain wave sensor 10 is attached to a user who is operating the information processing apparatus 20, and the brain wave sensor 10 is thereby capable of detecting the brain waves of the user. The brain wave sensor 10 may be any brain wave sensor capable of detecting the brain waves of the user. The brain wave sensor 10 may be an earphone-type brain wave sensor, but this example is not limiting.

The information processing apparatus 20 is an apparatus used by a user whose health condition is to be estimated. For example, if the user is a desk worker, the information processing apparatus 20 may be a device used by the user for work. The information processing apparatus 20 can estimate the health condition of the user by using the brain wave information acquired from the brain wave sensor 10.

The information processing apparatus 20 may be a general-purpose electronic device such as a PC (Personal Computer) or a tablet terminal.

The information processing apparatus 20 includes a communication interface 21, a memory 22, an input interface 23, an output interface 24, and a processor 25.

The communication interface 21 includes a communication module. For example, the communication interface 21 may include a communication module compatible with a LAN (Local Area Network). In one embodiment, the information processing apparatus 20 is connected to a network via the communication interface 21. The communication interface 21 may be capable of communicating with a server or the like via the network.

The memory 22 is, for example, a semiconductor memory, a magnetic memory, an optical memory, or the like, but is not limited to these. The memory 22 may function as, for example, a main memory device, an auxiliary memory device, or a cache memory. The memory 22 stores any information used in the operation of the information processing apparatus 20. For example, the memory 22 may store system programs, application programs, various types of information, and the like. A portion of the memory 22 may be installed outside the information processing apparatus 20. In this case, a portion of the externally installed memory 22 may be connected to the information processing apparatus 20 via any appropriate interface.

The input interface 23 includes one or more interfaces for input that receive an operation from the user and acquire input information based on the user operation. For example, the input interface 23 includes at least one of a keyboard, a mouse, and a touchscreen that is integrated with the display of the output interface 24 but is not limited to these.

The output interface 24 includes one or more interfaces for output that output information to notify the user. For example, the output interface 24 includes a display that outputs information as an image, a speaker that outputs information as sound, and the like, but is not limited to these.

The processor 25 is a general-purpose processor such as a central processing unit (CPU) or a graphics processing unit (GPU), or a dedicated processor that is dedicated to specific processing. The processor 25 may be a field-programmable gate array (FPGA), a digital signal processor (DSP), an application specific integrated circuit (ASIC), or the like. The processor 25 may be a combination of a plurality of processors. The processor 25 executes processes related to the operation of the information processing apparatus 20 while controlling each component of the information processing apparatus 20.

The functions of the information processing apparatus 20 can be realized by causing the processor 25 to execute a program stored in the memory 22. That is, the functions of the information processing apparatus 20 can be realized by software.

### Operation of health condition estimation system

Next, the operation of the health condition estimation system 1 will be described.

The brain wave sensor 10 detects the brain waves of a user who is operating the information processing apparatus 20. Upon detecting brain waves of the user, the brain wave sensor 10 transmits brain wave information to the information processing apparatus 20.

The processor 25 of the information processing apparatus 20 acquires the brain wave information transmitted by the brain wave sensor 10. When the brain wave sensor 10 and the communication interface 21 are communicating with each other, the processor 25 may acquire the brain wave information transmitted by the brain wave sensor 10 via the communication interface 21.

The processor 25 acquires operation information from the input interface 23 from when the user operates the input interface 23 of the information processing apparatus 20. Hereinafter, "operation information from when the user operates the input interface 23 of the information processing apparatus 20" may be simply referred to as "operation information".

The processor 25 may acquire various operations by the user on the input interface 23 as the operation information. In a case in which the input interface 23 includes a keyboard, for example, the processor 25 may acquire information such as typing speed and downtime as operation information. Also, in a case in which the input interface 23 includes a mouse, for example, the processor 25 may acquire information such as the mouse movement speed, number of clicks, and downtime as operation information.

The processor 25 may periodically acquire the operation information as time-series data. By acquiring operation information as time-series data, the processor 25 can recognize changes in the operation behavior of the user with respect to the input interface 23.

As illustrated in FIG. 1, the memory 22 stores an estimation model 221 and training data 222.

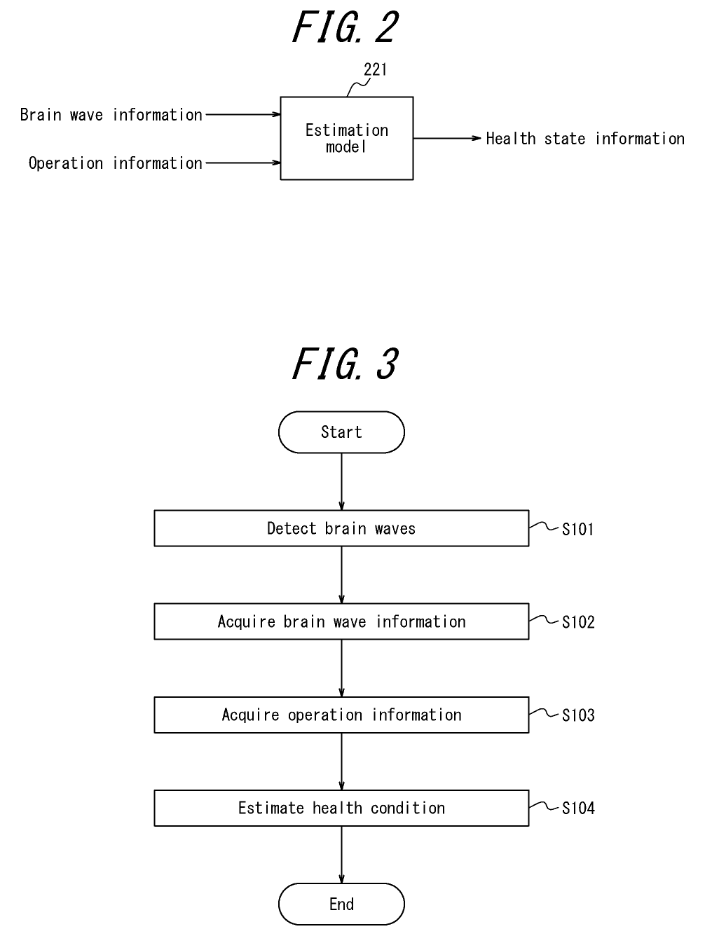

The estimation model 221 is a model for estimating the health condition of the user based on the brain wave information acquired from the brain wave sensor 10 and the operation information acquired from the input interface 23. As illustrated in FIG. 2, when the brain wave information and the operation information are inputted, the estimation model 221 estimates the health condition of the user and outputs information on the health condition of the user.

The estimation model 221 may be a model trained by machine learning based on the training data 222. The training data 222 is a dataset used by the estimation model 221 when performing machine learning. The training data 222 may be a dataset that associates past brain wave information and operation information for the user with past information on the health condition of the user. In a case in which the estimation model 221 is a model created by machine learning, the accuracy of estimating the health condition improves as the amount of accumulated past data increases.

The estimation model 221 is not limited to a model created by machine learning. The estimation model 221 may, for example, be a regression model.

In the example illustrated in FIG. 1, the memory 22 stores the training data 222, but this is just an example. The training data 222 may be stored in an apparatus other than the information processing apparatus 20, for example.

Upon acquiring the brain wave information from the brain wave sensor 10 and the operation information from the input interface 23, the processor 25 estimates the health condition of the user in real time based on the brain wave information and the operation information and outputs information on the health condition of the user. The processor 25 inputs the brain wave information and operation information into the estimation model 221 stored in the memory 22, thereby estimating the health condition of the user and outputting information on the estimated health condition of the user.

The information on the health condition of the user includes various types of information relating to the health condition. The information on the health condition may include, for example, the stress level of the user. The stress level of the user may, for example, be a numerical representation of the stress on the user.

Upon estimating the health condition of the user, the processor 25 may cause the output interface 24 to display information on the estimated health condition of the user. This enables users to understand information on their own health condition in real time.

The processor 25 may also generate an action proposal to propose to the user based on the information on the health condition outputted by the estimation model 221. For example, if the information on the health condition indicates that the user has a high stress level, an action proposal such as taking a break, stretching, exercising, or finishing work early may be generated.

Upon generating the action proposal, the processor 25 may cause the output interface 24 to display the action proposal. This enables users to adopt appropriate behaviors according to their own health condition and reduce their stress level.

When displaying the action proposal on the output interface 24, the processor 25 may display the action proposal together with the information on the health condition. This enables users to check their own health condition and appropriate behaviors at the same time.

When generating the action proposal, the processor 25 may acquire the schedule of the user and generate the action proposal based on the acquired schedule and the information on the health condition outputted by the estimation model 221. In this case, the processor 25 may acquire the schedule of the user from a terminal device such as a smartphone used by the user or may acquire the schedule of the user from a server that stores the schedule of the user.

For example, when the schedule of the user includes a meeting, the processor 25 may generate an action proposal to reduce the stress level before the start of the meeting, such as, "You have a meeting at 4 p.m. Why not take a break and refresh yourself before the meeting?"

This enables the processor 25 to generate appropriate action proposals according to the schedule of the user.

The operation of the health condition estimation system 1 will be described with reference to the flowchart illustrated in FIG. 3.

Step S101: the brain wave sensor 10 detects brain waves of the user who is operating the information processing apparatus 20. Upon detecting brain waves of the user, the brain wave sensor 10 transmits brain wave information to the information processing apparatus 20.

Step S102: the processor 25 of the information processing apparatus 20 acquires the brain wave information transmitted by the brain wave sensor 10.

Step S103: the processor 25 acquires operation information from the input interface 23 from when the user operates the input interface 23 of the information processing apparatus 20.

Step S104: upon acquiring the brain wave information from the brain wave sensor 10 and the operation information from the input interface 23, the processor 25 estimates the health condition of the user based on the brain wave information and the operation information. The processor 25 may cause the output interface 24 to display the information on the estimated health condition of the user.

According to the health condition estimation system 1 of the embodiment as described above, the estimation accuracy in estimating a health condition based on biological information can be improved. More specifically, the health condition estimation system 1 includes a brain wave sensor 10 that detects brain waves of the user and outputs brain wave information and an information processing apparatus 20. The information processing apparatus 20 includes a processor 25 configured to estimate a health condition of the user, based on the brain wave information and operation information from when the user operates the information processing apparatus 20, and output information on the health condition of the user. In this way, the information processing apparatus 20 estimates the health condition of the user by taking into consideration not only the brain wave information, which is the biological information for the user, but also the operation information from when the user operates the information processing apparatus 20. Therefore, according to the health condition estimation system 1 of the present embodiment, it is possible to improve the estimation accuracy in estimating a health condition based on biological information.

The health condition estimation system 1 according to the present embodiment estimates the health condition of the user based on the brain wave information and the operation information and therefore can detect stress in the user at an early stage.

The health condition estimation system 1 according to the present embodiment may generate an action proposal to propose to the user based on the information on the health condition and cause the generated action proposal to be displayed on the output interface 24. If the user responds by implementing the suggested action, the stress on the user can be reduced, and the concentration and productivity of the user can be improved.

It will be apparent to those skilled in the art that the present disclosure may be realized in certain forms other than the above embodiments without departing from the spirit or essential characteristics of the present disclosure. Accordingly, the foregoing description is merely illustrative and is not limiting. The scope of the disclosure is defined by the appended claims, not by the foregoing description. Among all modifications, those within a range of equivalents to the present disclosure shall be considered as being included in the present disclosure.

For example, the arrangement and number of each of the above-mentioned components are not limited to those illustrated in the above description and the drawings. The arrangement and number of each component may be configured freely as long as the corresponding function can be realized.

For example, a general-purpose electronic device such as a smartphone or a computer may be configured to function as the information processing apparatus 20 according to the above embodiment. Specifically, a program describing the processing details to realize each function of the information processing apparatus 20 according to the embodiment could be stored in the memory of the electronic device, and the program could be read and executed by the processor of the electronic device. An embodiment of the present disclosure can thus also be realized as a program executable by a processor.

For example, part of the processing executed by the information processing apparatus 20 in the above embodiment may be executed on a server capable of communicating with the information processing apparatus 20. For example, the information processing apparatus 20 may transmit the brain wave information and operation information to the server, and the server may execute processing to estimate the health condition of the user based on the brain wave information and operation information acquired from the information processing apparatus 20.

For example, in the above embodiment, the information processing apparatus 20 acquires brain wave information, but the information processing apparatus 20 may acquire biological information other than brain wave information together with the brain wave information. The biological information other than brain waves may, for example, be heart rate, blood pressure, or body temperature.

1. An information processing apparatus for estimating a health condition of a user, the information processing apparatus comprising:
a processor configured to estimate a health condition of the user, based on brain wave information of the user and operation information from when the user operates the information processing apparatus, and output information on the health condition of the user.

2. The information processing apparatus according to claim 1, further comprising:
a memory configured to store an estimation model created by machine learning based on training data, wherein
the estimation model is a model configured to output the information on the health condition when the brain wave information and the operation information are inputted, and
the processor is configured to output the information on the health condition by inputting the brain wave information and the operation information into the estimation model.

3. The information processing apparatus according to claim 1, further comprising:
an input interface configured to accept an operation by the user, wherein
the processor is configured to acquire, as the operation information, an operation by the user on the input interface.

4. The information processing apparatus according to claim 2, further comprising:
an input interface configured to accept an operation by the user, wherein
the processor is configured to acquire, as the operation information, an operation by the user on the input interface.

5. The information processing apparatus according to claim 3, wherein the input interface includes at least one of a keyboard, a mouse, and a touchscreen.

6. The information processing apparatus according to claim 3, further comprising:
an output interface, wherein
the processor is configured to generate an action proposal to propose to the user based on the information on the health condition and display the generated action proposal on the output interface.

7. The information processing apparatus according to claim 1, wherein the information on the health condition includes a stress level of the user.

8. The information processing apparatus according to claim 1, further comprising:
an output interface, wherein
the processor is configured to generate an action proposal to propose to the user based on the information on the health condition and display the generated action proposal on the output interface.

9. The information processing apparatus according to claim 8, wherein the processor is configured to acquire a schedule of the user and generate the action proposal based on the acquired schedule and the information on the health condition.

10. The information processing apparatus according to claim 9, wherein the information on the health condition includes a stress level of the user.

11. A health condition estimation system comprising:
the information processing apparatus according to claim 1; and
a brain wave sensor configured to detect brain waves of the user and output the brain wave information.

12. A health condition estimation method comprising:
acquiring brain wave information from a brain wave sensor configured to detect brain waves of a user; and
estimating a health condition of the user, based on the brain wave information and operation information from when the user operates an information processing apparatus, and outputting information on the health condition of the user.

13. A non-transitory computer readable medium having recorded thereon a program which, when executed by a computer, is configured to cause the computer to perform operations comprising:
acquiring brain wave information from a brain wave sensor configured to detect brain waves of a user; and
estimating a health condition of the user, based on the brain wave information and operation information from when the user operates an information processing apparatus, and outputting information on the health condition of the user.
